(19) United States
(12) Patent Application Publication
(10) Pub. No.: US 2011/0207685 A1
(43) Pub. Date: Aug. 25, 2011

(54) Oral Formulations of Chemotherapeutic Agents

(76) Inventors: David Bonnafous, Champigny Sur Marne (FR); Guy Cave, L'Hay Les Roses (FR); Assia Dembri, Paris (FR); Sophie Lebel-Binay, Villejuif (FR); Gilles Ponchel, Paris (FR); Emilienne Soma, Paris (FR)

(21) Appl. No.: 13/057,835

(22) PCT Filed: Aug. 6, 2009
(86) PCT No.: PCT/EP2009/060233

§ 371 Date: May 9, 2011

(30) Foreign Application Priority Data
Aug. 6, 2008 (EP) 08305455.1

(51) Int. Cl. A61K 31/704; A61K 31/4745; A61K 31/337; A61P 35/00; B82Y 5/00

(52) U.S. Cl. 514/34; 514/283; 514/449; 514/285; 977/773; 977/906

(57) ABSTRACT

The present invention is directed to new oral formulations of chemotherapeutic agents, their process of preparation as well as their therapeutic uses. More specifically, said invention is related to nanoparticles comprising at least one chemotherapeutic agents as an active ingredient, at least one polymer and at least one cyclic oligosaccharide capable of complexing said camptothecin derivative, said nanoparticles being for therapeutic oral administration.

### FIELD OF THE INVENTION

The present invention is directed to new oral formulations of chemotherapeutic agents, their process of preparation as well as their therapeutic uses.

### BACKGROUND OF THE INVENTION

Cancer is characterized by uncontrolled growth of cells coupled with malignant behavior: invasion and metastasis. It is a major cause of mortality in most industrialized countries. Different ways of cancer treatment can be used: chemotherapy, radiotherapy, surgery, immunotherapy and hormonotherapy.

Chemotherapy can be defined as the use of cytotoxic drugs (also named “chemotherapeutic agents”), to treat cancer. Broadly, most chemotherapeutic agents work by impairing mitosis (cell division) or DNA synthesis, effectively targeting fast-dividing cells. As these drugs cause damage to cells they are termed “cytotoxic”.

Chemotherapeutic agents are delivered most often parenteraly, noteworthy intravenously (i.v.). Capecitabine, Tegafur and Navelbine are examples of only few chemotherapeutic agents orally administered. Intravenous chemotherapy can be given over different amounts of time, depending on the drug and the type of cancer to be treated. For instance, the drugs for each course of chemotherapy may be given to patients as
an injection into a vein, over a few minutes,
through a drip (intravenous infusion) over anything from 30 minutes to a few hours,
through a drip or pump over 2 or more days, through a pump that the patient has to wear for weeks or months; Chemotherapy given over weeks or months is called a ‘continuous infusion’. It is also called “protracted venous infusion” (PVI) or ‘ambulant infusion’ (this means that the patient must walk around wearing the pump).

If chemotherapeutic treatment only lasts a few hours, the patient usually has to spend a day at the hospital to receive the appropriate treatment from doctor or specially trained nurse. If the treatment takes longer than a few hours, the patient often needs to be admitted to a ward at the hospital.

Thus, parenteral administration of chemotherapeutic agents is associated with some disadvantages and drawbacks, including patient discomfort, like pain or fear of needle injection. As the patient is not able to self-administer the chemotherapeutic agent, he needs to travel to the physician's office for drug administration, with obvious patient's inconvenience.

Therefore, oral administration of chemotherapeutic agent presents several advantages like patient's preference, convenience, reduced hospital stay, and reduced cytotoxic exposure risk for health care workers,

WO 99/43359 discloses nanoparticles comprising at least (i) one polymer, (ii) one cyclic oligosaccharide and (iii) one active ingredient. It has been described that these nanoparticles allow sustained (controlled) release of drug (Maincent P and al “Preparation and in vivo studies of a new drug delivery system. Nanoparticles of alkylcyanoacrylate”, Appl. Biochem. Biotechnol. 1984;10:263-5) and have bioadhesion properties, noteworthy in the gastrointestinal tract (Ponchel G and al. “Specific and non-specific bioadhesive particulate systems for oral delivery to the gastrointestinal tract”, Adv Drug Deliv Rev. 1998 Dec 1;34(2-3):191-219). Said Nanoparticles are hereinafter referred to as “Sustained Released Nanoparticles” (“SRN”).

The inventors have now surprisingly found that SRN containing chemotherapeutic agents are suitable for oral administration (hereinafter referred to as “oral formulations of the invention”).

Besides several advantages of oral administration in general (e.g., patient's preference, convenience, reduced hospital stay, and reduced cytotoxic exposure risk for health care workers), more specific advantages can be identified. Indeed, oral formulations of the invention allow overcoming limits of intravenously or orally administered chemotherapeutic agents.

Chemotherapy can be physically exhausting for the patient because of its cytotoxicity on non-tumourous cells. Current intravenous or oral chemotherapeutic treatments have a range of side effects, depending on the chemotherapeutic agent. Common side effects include pain, nausea and/or vomiting, diarrhea or constipation, anemia, malnutrition, hair loss, memory loss, depression of the immune system, hence (potentially lethal) infections and sepsis, dehydration, vertigo, hematoma, dry mouth/xerostomia, psychosocial distress, weight loss or gain, water retention, hemorrhage, kidney damage, secondary neoplasms, cardiotoxicity, hepatotoxicity, nephrotoxicity, neurotoxicity, sexual impotence, ototoxicity, . . .

The inventors have now surprisingly found that oral formulations of the invention can reduce side effects of chemotherapeutic agents and can thus improve their tolerance.

Further, development of oral chemotherapeutic treatments is limited by their unsuitable bioavailability, compared to intravenous route. Bioavailability is a measurement of the rate and extent of a therapeutically active drug to reach the systemic circulation and to be available at the site of action. The inventors have also demonstrated that oral formulations of the invention improve the bioavailability of chemotherapeutic agents.

Several non limiting examples showing the advantages of oral formulation of the invention are given below.

Camptothecin (CPT) is a hydrosoluble cytotoxic quinoline alkaloid family isolated from Camptotheca acuminata (Camptotheca, Happy tree), a tree native in China and Tibet. It inhibits the DNA enzyme topoisomerase I. Topoisomerase I, an intranuclear enzyme that noncovalently binds to torsionally strained, supercoiled, double-stranded DNA, creates a transient single-strand break (named “cleavable complex”) in the DNA molecule. This allows for the passage of an intact complementary DNA strand during replication, transcription, recombination, and other DNA functions. The enzyme-bridged DNA breaks, also known as cleavable complexes, are then resealed by the topoisomerase I enzyme. Dissociation of the enzyme restores an intact, newly relaxed DNA double helix.

CPT stabilize the cleavable complex between the topoisomerase I molecule and the free 3′-phosphate of the DNA. The resulting enzyme-linked DNA breaks induce cytotoxicity. In the past, CPT has often been referred to as topoisomerase I inhibitor. However, it is not classic enzyme inhibitor since rather than directly altering the function of topoisomerase I, it converts this normal endogenous protein into a cellular toxin. Therefore, CPT is often preferentially referred to as topoisomerase I poison (Bomgaars and al. (2001) Oncologist 6(6): 506-16).

CPT showed anticancer activity in preliminary clinical trials and two CPT analogs have been approved and are used in cancer chemotherapy: (i) irinotecan (CPT-11), marketed as Campto or Camptosar by UpJohn (now Pfizer) and (ii) topotecan, marketed as Hymcamptamin, Hycamptin, or Thycantin, by Smith Kline & Beecham (now GSK) (Ulukan, and al. (2002) Drugs 62 (2): 2039-2057). Other CPT analogs include hexatecan, silatecan, lutortecan, karenitecin (BNP1350), gimatecan (ST1481), belotecan (CKD602) or their pharmaceutically acceptable salts. However CPT analogs present significant drawbacks as low solubility and adverse effect.

Irinotecan (“IRN” or “CPT-11” or “Irinotecan hydrochloride”) is a semisynthetic analogue of camptothecin and a well-known anticancer chemotherapeutic agent with a main indication in metastatic colorectal cancer. It is also studied for the treatment of lung cancers, stomach cancer, pancreas cancer, non-Hodgkin lymphoma, uterine cervix cancer, head and neck cancers, brain cancer and ovary cancer.

Irinotecan is a prodrug that is converted by carboxylesterase in the liver, intestinal tract, and some tumors to its active metabolite, SN-38 (7-ethyl10-hydroxycamptothecin). SN-38 is 100- to 1,000-fold more potent than irinotecan. SN-38 is then inactivated by glucuronidation by uridine diphosphate glucuronosyltransferases (UGT), to form SN-38 glucuronide (SN-38G or 7-ethyl-10-[3,4,5-trihydroxy-pyran-2-carboxylic acid]camptothecin) (Kawato et al., 1991 Cancer Chemother Pharmacol 28(3): 192-8; Takasuna et al., Cancer Res (1996) 56(16): 3752-7; Slatter et al., 1997 Metab Dispos 25(10): 1157-64)).

A drawback of irinotecan is severe adverse effects and notably severe diarrhea and extreme suppression of the immune system. Irinotecan-associated diarrhea may be clinically significant, sometimes leading to severe dehydration requiring hospitalization or intensive care unit admission. Irinotecan-associated suppression of the immune system leads to dramatically lowered white blood cell counts in the blood, in particular the neutrophils. While the bone marrow, where neutrophils are made, cranks up production to compensate, the patient may experience a period of neutropenia, that is, a clinical lack of neutrophils in the blood.

The efficacy of Irinotecan is known to be dose-dependent and has also been shown to be schedule-dependent, with prolonged low-dose administration being more effective and less toxic than short duration high-dose schedules (Houghton, P. J. et. al. “Efficacy of Topoisomerase I Inhibitors Topotecan and Irinotecan administered at Low Dose Levels in Protracted Schedules to Mice Bearing Xenografts of Human Tumors” Cancer Chemother. Pharmacol. (1995), 36, 393-403; Thompson, J. et. al. “Efficacy of Systemic Administration of Irinotecan Against Neuroblastoma Xenografts” Clin. Cancer Res. (1997), 3, 423-432; Furman W L and al, “Direct translation of a protracted irinotecan schedule from a xenograft model to a phase I trial in children” Journal of clinical oncology (1999), 17, 1815-1824).

An efficient approach to prolong exposure is to use the oral route. Besides several advantages of oral administration in general (e.g., patient's preference, convenience, reduced hospital stay, and reduced cytotoxic exposure risk for health care workers), more specific advantage can be distinguished. Indeed, compared with i.v. administration, the metabolic ratio of total SN-38 to total irinotecan after oral administration is higher (Gupta E and al. Pharmacokinetic and pharmacodynamic evaluation of the topoisomerase inhibitor irinotecan in cancer patients. J Clin Oncol 1997;15:1502-10).

Thus the need has arisen to develop oral formulations of camptothecin derivatives that would improve bioavailability and ameliorate side effects. Up to date, oral administration of free camptothecin derivatives and development of oral IRN formulations are limited by the lack of tolerance, notably a severe diarrhea. Moreover, the oral bioavailability of irinotecan is reported to be only about 20% compare to its i. v. bioavailability. Thus, serious problems of absorption and pre-systemic metabolism of irinotecan need to be overcome before oral delivery becomes available as a treatment option.

It is therefore desirable to provide new formulations of Irinotecan allowing tolerable and bioavailable oral administration.

The inventors have now surprisingly found that SRN containing camptothecin derivatives (“camptothecin-SRN”) are suitable for oral administration of camptothecin derivatives. They have surprisingly shown that oral administration of camptothecin-SRN is better tolerated than intravenous and oral free camptothecin without loss of anti-tumor efficacy. Moreover, oral administration of camptothecin-SRN increase its bioavailability, compared to intravenous or oral free camptothecin solution. The SRN are thus highly advantageous in connection with camptothecin derivatives such as, but not only, irinotecan (irinotecan-SRN).

Indeed, in a non obviously way, camptothecin-SRN allow oral administration and sustained release of active ingredient, which help to improve its bioavailability and reduce its side effects. The bioadhesion of SRN on intestinal mucous membrane increases the residence time and thus reinforce the sustained release of the active ingredient.

Moreover, improving irinotecan residence time in the gastro-intestinal tract allows a higher conversion of irinotecan into its active metabolite SN38. In particular, it was found that the IRN-SRN were able to efficiently protect the active agent from the pH-dependent degradation such as in the gastro-intestinal environment. As a result, the Irinotecan's lactone form is protected from conversion to its inactive form (carboxylate) which would normally occur with oral administration, as IRN and SN38 are instable in the intestinal pH (pH 5,5 to 7).

Nanoparticles may be insufficiently concentrated and would constrain the patient to swallow too important volumes of SRN. Thus the oral formulations of the invention are advantageously concentrated in a volume suitable for orally administration to the patient.

The inventors have identified a lyophilization, also called freeze-drying, process to overcome this drawback. In particular, they have also determined a formulation of chemotherapeutic agent-SRN suitable for said freeze-drying process. The freeze-drying process and said formulations are further objects of the present invention. They are particularly appropriate for industrialization.

Doxorubicin (also known as hydroxydaunorubicin) is a hydrosoluble drug used in cancer chemotherapy. It is an anthracycline antibiotic, closely related to the natural product daunomycin. Doxorubicin is commonly used to treat some leukemias, Hodgkin's lymphoma, as well as cancers of the bladder, breast, stomach, lung, ovaries, thyroid, soft tissue sarcoma, multiple myeloma, and others.

Doxorubicin is known to interact with DNA by intercalation and inhibition of macromolecular biosynthesis. This inhibits the progression of the enzyme topoisomerase II, which unwinds DNA for transcription. Doxorubicin stabilizes the topoisomerase II complex after it has broken the DNA chain for replication, preventing the DNA double helix from being resealed and thereby stopping the process of replication.

Acute side-effects of doxorubicin can include nausea, vomiting, and heart arrhythmias. It can also cause neutropenia (a decrease in white blood cells), as well as complete alopecia (hair loss). When the cumulative dose of doxorubicin reaches 550 mg/m², the risks of developing cardiac side effects, including congestive heart failure, dilated cardiomyopathy, and death, dramatically increase. Doxorubicin cardiotoxicity is characterized by a dose-dependent decline in mitochondrial oxidative phosphorylation. Reactive oxygen species, generated by the interaction of doxorubicin with iron, can then damage the myocytes (heart cells), causing myofibrillar loss and cytoplasmic vacuolization. Additionally, some patients may develop palmar plantar erythrodysesthesia, or, “hand-foot syndrome,” characterized by skin eruptions on the palms of the hand or soles of the feet, characterized by swelling, pain and erythema. Doxorubucin can also cause reactivation of hepatitis B.

Doxorubicin is only intravenously administered to patients. Thus, there is a need of an oral formulation of doxorubicin. Doxorubicin oral formulation is limited by a toxic (necrotic) action of doxorubicin on the gastrointestinal tract. Moreover doxorubicin oral effectiveness is limited by pre-systemic deactivation in the gastrointestinal tract, leading to an unsuitable bioavailability. Indeed, oral bioavailability of doxorubicin is only about 5% compare to its i. v. bioavailability. Inventors have surprisingly shown that doxorubicin oral formulations of the invention is well tolerated and bioavailability of doxorubicin should be improved by oral formulations of the invention.

Paclitaxel and docetaxel are hydrophobic mitotic inhibitors used in cancer chemotherapy. They together belong to the category of the taxanes. Paclitaxel is still produced by isolation from natural sources while docetaxel, a semi-synthetic analogue of paclitaxel, is synthesized from 10-deacetyl baccatin. Paclitaxel differs from docetaxel by an acetylated hydroxyl function at position 10 and a benzoyl moiety instead of tert-butyl on the phenylpropionate side chain. Thus, paclitaxel and docetaxel have very close chemical formula and physicochemical properties. The mechanism of action of taxanes is based on their ability to bind to the β subunit of tubulin which interferes with the depolymerization of microtubules, thereby damaging dividing cells. This specificity of action is widely used in oncology to treat different solid tumors, especially ovarian, lung, breast, bladder, head and neck cancer.

Common side-effects include nausea and vomiting, loss of appetite, change in taste, thinned or brittle hair, pain in the joints of the arms or legs lasting 2-3 days, changes in the color of the nails, tingling in the hands or toes. More serious side effects such as unusual bruising or bleeding, pain/redness/swelling at the injection site, change in normal bowel habits for more than 2 days, fever, chills, cough, sore throat, difficulty swallowing, dizziness, shortness of breath, severe exhaustion, skin rash, facial flushing and chest pain can also occur. A number of these side effects are associated with the excipient used, Cremophor EL, a polyoxyethylated castor oil. Allergies to drugs such as cyclosporine, teniposide and drugs containing polyoxyethylated castor oil may indicate increased risk of adverse reactions to paclitaxel.

Paclitaxel and docetaxel have poor oral bioavailability. Scarce water solubility also makes it impossible to use oral solutions of taxanes. Indeed, aqueous dispersibility is a central problem that therefore must be overcome in order to prepare an oral dosage form for hydrophobic drugs, like paclitaxel or docetaxel. Therefore, intravenous (i.v.) infusion is the only way of administration. Three intravenous pharmaceutical compositions are today commercially available:
TAXOL® (active principle: paclitaxel) is based on the ability of CREMOPHOR®, a polyethoxylated castor oil, to dissolve paclitaxel in the weight-to-weight (w/w) ratio 87:1. It is chronologically the first commercial taxane formulation which has opened the era of taxane use in oncology. However it was later found that CREMOPHOR® is the cause of hypersensitivity reactions during TAXOL® infusion and for minimizing the incidence and severity of these reactions, a premedication with histamine blockers and glucocorticoids as well as continuous infusion schedules became standard practice.
TAXOTERE® is formulated from 40 mg/mL-1 of docetaxel and 1040 mg/mL-1 of polysorbate 80 (Tween 80), a surfactant of low molar mass made up of a nonionic polar group connected to a hydrocarbon segment, the major component of which is polyoxyethylene sorbitan monooleate which has a structure similar to that of polyethyleneglycol. This makes it highly soluble in water. From the first clinical trials, it was observed that intravenous administration of such formulations was accompanied by more or less severe hypersensitivity reactions, ranging from mild pruritis to anaphylactic shock, as well as considerable fluid retention reflected as weight gain, peripheral oedema and, occasionally, pleural and pericardial effusions. The hypersensitivity reactions, with an incidence rate of between 5% and 60%, have been attributed to the excipient used, polysorbate 80, and more specifically to the oxidation products and oleic acid present in polysorbate 80, known to cause the release of histamine responsible for these hypersensitivity reactions. That is why it is often necessary to pre-medicate with corticosteroids and antihistamines to avoid side effects. Moreover, dilution of the preparation in ethanol is required prior to administration of the formulation, which is clearly a major disadvantage for the patient to be treated.
ABRAXANE®, a third delivery system, consists of paclitaxel nanoparticles stabilized by human serum albumin in the w/w ratio 9:1 with the mean diameter of nanoparticles being 130 nm. The absence of non-ionic surfactants simplifies the treatment as no premedication is necessary and the infusion time is shortened. On the other hand the ABRAXANE® formulation is less potent than TAXOL® because ABRAXANE® nanoparticles like other particles with the size more than 100 nm are a substrate for reticuloendothelial system. Another disadvantage of this drug delivery vehicle is that human serum albumin isolated from donor blood is used, which always carries a small but definite risk of transmission of viral diseases.

Consequently, the need has arisen for a more bioavailable, less toxic and better tolerated dissolved paclitaxel or docetaxel formulations.

Inventors have now surprisingly shown that bioavailability of orally administered taxanes is significantly improved when formulated as taxanes-SRN.

The oral formulations of the invention also allow a better solubilization of taxanes. Indeed, cyclodextrins contained in the SRN are able to complex the active ingredient. As described in international patent application WO/9943359, SRN enables the active ingredient, even if it is hydrophobic, amphiphilic and/or insoluble, to penetrate inside the polymer structure resulting from association of the polymer or polymers and cyclodextrin or cyclodextrins, with an encapsulation yield within this structure that is significantly increased compared with the prior art. The yield appears to be related to the equilibrium between, firstly, solubilisation resulting from use of compounds able to complex the active ingredient (cyclodextrin) and, secondly, affinity of the active ingredient for the new polymer structure, which brings substantial progress at therapeutic and industrial levels. Thus, SRN allows loading nanoparticles, not only with hydrophilic active ingredients, but also with hydrophobic, amphiphilic and/or insoluble active ingredients.

More particularly, surprising solubilization efficacies were obtained by specific synergistic mixture of cyclodextrines. Indeed, the mixtures of Hydroxypropyl-β-cyclodextrin (HP-βCD) with Methylated-β-cyclodextrin (Me-β-CD); HP-βCD with γ-CD; or Me-β-CD with γ-CD allow to significantly improve solubility of active ingredients, such as taxanes.

The formulations of a taxane in a mixture of Hydroxypropyl-β-cyclodextrin (HP-βCD) with Methylated-β-cyclodextrin (Me-β-CD); or HP-βCD with γ-CD; or Me-β-CD with γ-CD are also parts of the present invention.

### SUMMARY OF THE INVENTION

The present invention concerns nanoparticles comprising at least one chemotherapeutic agent as an active ingredient, at least one polymer and at least one cyclic oligosaccharide capable of complexing said chemotherapeutic agent, for therapeutic oral administration of said chemotherapeutic agent derivatives.

Said nanoparticles comprising said chemotherapeutic agent are herein called “chemotherapeutic agent-SRN”.

The present invention also concerns said chemotherapeutic agent-SRN for the treatment and/or the prevention of cancer, said treatment and/or prevention comprising the oral administration of said chemotherapeutic agent-SRN.

Preferably, said polymer is chosen from the poly(alkylcyanoacrylate) in which the alkyl group, linear or branched, comprises one to twelve carbon atoms.

Preferably, said polymer is the poly(isohexylcyanoacrylate). This polymer may be obtained from polymerisation of Monorex® (Bioalliance Pharma).

Preferably, said cyclic oligosaccharide is a cyclodextrin, such as neutral or charged cyclodextrin, native (cyclodextrins 60 , β, γ, δ, ε), branched or polymerised or chemically modified. It is preferably a chemically modified cyclodextrin, by substituting one or more hydroxy groups with alkyl, aryl, arylalkyl, glycoside groups or by etherification, esterification with alcohols or aliphatic acids. More preferred cyclodextrins can be selected among, but not only, Hydroxypropyl-β-cyclodextrin or HP-βCD (available from Roquette), Randomly Methylated-β-cyclodextrin or Rameb-CD (available from Cyclolab), Methylated-β-cyclodextrin or Me-β-CD, sulfobutylether-β-cyclodextrin or Captisol (available from Cydex), γ-CD.

The nanoparticles of the invention may additionally comprise further pharmaceutically acceptable excipients as those generally used in the field, such as surfactives, stabilizing agents or tensioactives such as dextran or poloxamer or other non ionic surfactive agents (like polysorbate, sorbitan esters or others). Poloxamers are preferred, such as Poloxamer 188 (also named pluronic F68).

The size of the nanoparticles generally depends on the concentration in the cyclic oligosaccharide capable of complexing the active principle, the pH of the polymerization medium, and the stirring rate. The size of the nanoparticles is lower than 1 micrometer, preferably comprised between 20 nm to 1000 nm and more preferably between 50 nm to 700 nm.

It is thus possible by conducting standard preliminary tests to adjust the size of the nanoparticles, depending of the particularly desired effect.

The nanoparticles of the present invention preferably comprise, in weight, percentage:
from 0.1 to 30% of said chemotherapeutic agent;
from 10 to 85% of said polymer;
from 0.1 to 70% of said cyclic oligosaccharide capable of complexing said chemotherapeutic agent.

Additionally, the nanoparticles of the invention may also comprise:
from 0 to 60% of excipients, such as poloxamer(s); and/or
from 0 to 2% of acid(s), such as citric acid.

Preferred nanoparticles according to the invention include those comprising:
at least one chemotherapeutic agent chosen from irinotecan, doxorubicine, paclitaxel, docetaxel, ellipticine or their pharmaceutically acceptable salts;
poly(isohexylcyanoacrylate);
Poloxamer 188; and
hydroxypropyl-β-cyclodextrin and/or rameb methylated-β-cyclodextrin and/or methylated-β-cyclodextrin and/or γ-cyclodextrin.

According to the present invention, chemotherapeutic agents can refer to (i) topoisomerase inhibitors, (ii) anthracyclines, (iii) spindle poison plant alkaloids, (iv), alkylating agents, (v) anti-metabolites, and (vi) other chemotherapeutic agents:

### (i) Topoisomerase Inhibitors,

Topoisomerases are essential enzymes that maintain the topology of DNA. Inhibition of type I or type II topoisomerases interferes with both transcription and replication of DNA by upsetting proper DNA supercoiling. Some type I topoisomerase inhibitors include camptothecins derivatives Camptothecin derivatives refer to camptothecin analogs such as irinotecan, topotecan, hexatecan, silatecan, lutortecan, karenitecin (BNP1350), gimatecan (ST1481), belotecan (CKD602), . . . or their pharmaceutically acceptable salts. Irinotecan, its active metabolite SN38 and topotecan are preferred. Irinotecan is more preferred.

Examples of type II topoisomerase inhibitors include amsacrine, etoposide, etoposide phosphate, teniposide . . . These are semisynthetic derivatives of epipodophyllotoxins, alkaloids naturally occurring in the root of American Mayapple (Podophyllum peltatum).

### (ii) Anthracyclines:

Anthracyclines (or anthracycline antibiotics) are derived from Streptomyces bacteria. These compounds are used to treat a wide range of cancers, including leukemias, lymphomas, and breast, uterine, ovarian, and lung cancers. Anthracyclines have three mechanisms of action:

Inhibition of DNA and RNA synthesis by intercalating between base pairs of the DNA/RNA strand, thus preventing the replication of rapidly-growing cancer cells.

Inhibition topoiosomerase II enzyme, preventing the relaxing of supercoiled DNA and thus blocking DNA transcription and replication.

Creation of iron-mediated free oxygen radicals that damage the DNA and cell membranes.

Some non limitating examples of anthracyclins are: doxorubicin daunorubicin, epirubicin, idarubicin, valrubicin or their pharmaceutically acceptable salts.
(iii) Spindle Poison Plant Alkaloids

These alkaloids are derived from plants and block cell division by preventing microtubule function, essential for cell division.

Examples are vinca alkaloids (like vinblastine, vincristine, vindesine vinorelbine vinpocetine . . . ) and taxanes. Taxanes include paclitaxel and docetaxel or their pharmaceutically acceptable salts. Paclitaxel was originally derived from the Pacific yew tree. Docetaxel is a semi-synthetic analogue of paclitaxel. In contrast to the taxanes, the vinca alkaloids destroy mitotic spindles. Both taxanes and vinca alkaloids are therefore named spindle poisons or mitosis poisons, but they act in different ways.

### (iv) Alkylating Agents:

Alkylating agents are so named because of their ability to add alkyl groups to many electronegative groups under conditions present in cells. They impair cell function by forming covalent bonds with the amino, carboxyl, sulfhydryl, and phosphate groups in biologically important molecules. Noteworthy, their cytotoxicity is thought to result from inhibition of DNA synthesis.
Platinum compounds like oxaliplatin, cisplatin, carboplatin . . . are alkylating agents. Other alkylating agents are mechlorethamine, cyclophosphamide, chlorambucil, ifosfamide.

### (v) Anti-Metabolites:

An anti-metabolite is a chemical that inhibits the use of a metabolite, which is part of normal metabolism. Such substances are often similar in structure to the metabolite that they interfere with. The presence of anti-metabolites halters cell growth and cell division,
Purine or pyrimidine analogues prevent the incorporation of nucleotides into DNA, stopping DNA synthesis and thus cell divisions. They also affect RNA synthesis. Examples of purine analogues include azathioprine, mercaptopurine, thioguanine, fludarabine, pentostatin and cladribine . . . Examples of pyrimidine analogues include 5-fluorouracil (5FU), which inhibits thymidylate synthase, floxuridine (FUDR) and cytosine arabinoside (Cytarabine) . . .
Antifolates are drugs which impair the function of folic acids. Many are used in cancer chemotherapy, some are used as antibiotics or antiprotozoal agents. A well known example is Methotrexate. This is a folic acid analogue, and owing to structural similarity with it binds and inhibits the enzyme dihydrofolate reductase (DHFR), and thus prevents the formation of tetrahydrofolate. Tetrahydrofolate is essential for purine and pyrimidine synthesis, and this leads to inhibited production of DNA, RNA and proteins (as tetrahydrofolate is also involved in the synthesis of amino acids serine and methionine). Other antifolates include trimethoprim, raltitrexed, pyrimethamine and pemetrexed . . .

### (vi) Other Chemotherapeutic Agents

Examples above are not limitating and other chemotherapeutic agents can be described.
Among others, ellipticine and harmine can be cited.
Ellipticine is a natural plant alkaloid product which was isolated from the evergreen tree of the Apocynaceae family. Ellipticine was found to have cytotoxic and anticancer activity (Dalton et al., Aust. J. Chem., 1967. 20, 2715). The ellipticine derivative hydroxylated in position 9 (9-hydroxyellipticinium) was found to have greater antitumoural activity than ellipticine on many experimental tumours (Le Pecq et al., Proc. Natl. Acad, Sci., USA, 1974, 71, 5078-5082). Researches were performed to identify an ellipticine derivative appropriate for human therapeutics and lead to the preparation of Celiptium, or N2-methyl-9-hydroxyellipticinium (NMHE), which has been used for the treatment of some human cancers, in particular for the treatment of bone metastasis of breast cancers. Other 9-hydroxy ellipticine derivatives, such as 2-(diethyiamino-2-ethyl)9-hydroxyellipticinium acetate, 2-(diisopropylamino-ethyl)9-hydroxy-ellipticinium acetate and 2-(beta piperidino-2-ethyl)9-hydroxyellipticinium, had been described for instance in the US patent U.S. Pat. No. 4,310,667.
Harmine is a natural plant alkaloid product which was isolated from the Peganum harmala seeds. Peganum harmala (Zygophyllaceae) is a plant widely distributed in semi arid rangelands in the Central Asia, North Africa, Middle East and Australia. The pharmacologically active compounds of P. harmala are several alkaloids that are found especially in the seeds and the roots. These incude (3-carbolines such as harmine, harmaline, harmol, harmalol and harman, and quinazoline derivatives: vasicine and vasicinone.
Peganum harmala alkaloids were found to possess significant antitumour potential (Lamchouri and al., Therapie, 1999, 54(6):753-8). Proliferation of tumoral cells lines was significantly reduced
Harmine was reported to exhibit strong cytotoxicity against a number of human tumor cell lines (Ishida and al, Bioorg Mad Chem Lett, 1999, 9(23):3319-24). Anticancer activity of harmol dimers has also been described for instance in the international patent WO2009047298.

Cancer herein refers to any malignant proliferative cell disorders such as tumour or leukemia, including carcinoma, sarcoma, lymphoma, stem cell tumor, blastoma and include any kind of colorectal, prostate, lung, stomach, pancreas, uterine cervix, head and neck, brain, breast and ovary cancers, non-Hodgkin lymphoma, leukemia.

As used herein, the term “patient” refers to either an animal, such as a valuable animal for breeding, company or preservation purposes, or preferably a human or a human child, which is afflicted with, or has the potential to be afflicted with one or more diseases and conditions described herein.

As used herein, a “therapeutically effective amount” refers to an amount of a compound of the present invention which is effective in preventing, reducing, eliminating, treating or controlling the symptoms of the herein-described diseases and conditions. The term “controlling” is intended to refer to all processes wherein there may be a slowing, interrupting, arresting, or stopping of the progression of the diseases and conditions described herein, but does not necessarily indicate a total elimination of all disease and condition symptoms, and is intended to include prophylactic treatment.

As used herein, the term “pharmaceutically acceptable” refers to those compounds, materials, excipients, compositions or dosage forms which are, within the scope of sound medical judgment, suitable for contact with the tissues of human beings and animals without excessive toxicity, irritation, allergic response or other problem complications commensurate with a reasonable benefit/risk ratio.

As used herein, “pharmaceutically acceptable salts” refer to derivatives of the disclosed compounds wherein the parent compound is modified by making acid or base salts thereof. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, tartaric, citric, methanesulfonic, benzenesulfonic, glucoronic, glutamic, benzoic, salicylic, toluenesulfonic, oxalic, fumaric, maleic, lactic and the like. Further addition salts include ammonium salts such as tromethamine, meglumine, epolamine, etc., metal salts such as sodium, potassium, calcium, zinc or magnesium.

The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. Generally, non-aqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418, the disclosure of which is hereby incorporated by reference.

As used herein, “pharmaceutically acceptable excipient” includes any carriers, diluents, adjuvants, or vehicles, such as preserving or antioxidant agents, fillers, disintegrating agents, wetting agents, emulsifying agents, suspending agents, solvents, dispersion media, coatings, antibacterial and antifungal agents, isotonic and absorption delaying agents and the like. The use of such media and agents for pharmaceutical active substances is well-known in the art. Except insofar as any conventional media or agent is incompatible with the active ingredient, its use in the therapeutic compositions is contemplated. Supplementary active ingredients can also be incorporated into the compositions as suitable therapeutic combinations.

The present invention also concerns the corresponding methods of treatment comprising the oral administration of a nanoparticle of the invention together with a pharmaceutically acceptable excipient to a patient in the need thereof.

The identification of those subjects who are in need of treatment of herein-described diseases and conditions is well within the ability and knowledge of one skilled in the art. A veterinarian or a physician skilled in the art can readily identify, by the use of clinical tests, physical examination, medical/family history or biological and diagnostic tests, those subjects who are in need of such treatment.

A therapeutically effective amount can be readily determined by the attending diagnostician, as one skilled in the art, by the use of conventional techniques and by observing results obtained under analogous circumstances. In determining the therapeutically effective amount, a number of factors are considered by the attending diagnostician, including, but not limited to: the species of subject; its size, age, and general health; the specific disease involved; the degree of involvement or the severity of the disease; the response of the individual subject; the particular compound administered; the mode of administration; the bioavailability characteristic of the preparation administered; the dose regimen selected; the use of concomitant medication; and other relevant circumstances.

The amount of the chemotherapeutic agent, which is required to achieve the desired biological effect, will vary depending upon a number of factors, including the chemical characteristics (e.g. hydrophobicity) of the compounds employed, the potency of the compounds, the type of disease, the species to which the patient belongs, the diseased state of the patient, the route of administration, the bioavailability of the compound by the chosen route, all factors which dictate the required dose amounts, delivery and regimen to be administered.

In the context of the invention, the term “treating” or “treatment”, as used herein, means reversing, alleviating, inhibiting the progress of, or preventing the disorder or condition to which such term applies, or one or more symptoms of such disorder or condition.

In general terms, the compounds of this invention may be provided in an aqueous solution or suspension containing 0.05 to 10% w/v compound. Typical dose ranges are from 1 μg/kg to 0.1 g/kg of body weight per day; a preferred dose range is from 0.01 mg/kg to 10 mg/kg of body weight per day or an equivalent dose in a human child. The preferred dosage of drug to be administered is likely to depend on such variables as the type and extent of progression of the disease or disorder, the overall health status of the particular patient, the relative biological efficacy of the compound selected, the formulation of the compound, the pharmacokinetic properties of the compound by the chosen delivery route, schedule of administrations (number of repetitions in a given period of time), and concomitant treatment.

The compounds of the present invention are also capable of being administered in unit dose forms, wherein the term “unit dose” means a single dose which is capable of being administered to a patient, and which can be readily handled and packaged, remaining as a physically and chemically stable unit dose comprising either the active compound itself, or as a pharmaceutically acceptable composition, as described hereinafter. As such, typical total daily dose ranges are from 0.01 to 100 mg/kg of body weight. By way of general guidance, unit doses for humans range from 1 mg to 3000 mg per day. Preferably, the unit dose range is from 1 to 1000 mg administered one to six times a day, and even more preferably from 10 mg to 1000 mg, once a day. Compounds provided herein can be formulated into pharmaceutical compositions by admixture with one or more pharmaceutically acceptable excipients. Such unit dose compositions may be prepared for use by oral administration, particularly in the form of tablets, capsules, oral suspension, powder to resuspend or syrup.

The compositions may conveniently be administered in unit dosage form and may be prepared by any of the methods well-known in the pharmaceutical art, for example, as described in Remington: The Science and Practice of Pharmacy, 20^{th} ed.; Gennaro, A. R., Ed.; Lippincott Williams & Wilkins: Philadelphia, Pa., 2000.

For oral administration, tablets, pills, powders, capsules, suspension, syrup and the like can contain one or more of any of the following vehicles, or compounds of a similar nature: a binder such as microcrystalline cellulose, or gum tragacanth; a diluent such as starch or lactose; a disintegrant such as starch and cellulose derivatives; a lubricant such as magnesium stearate; a glidant such as colloidal silicon dioxide; a sweetening agent such as sucrose or saccharin; or a flavoring agent. Capsules can be in the form of a hard capsule or soft capsule, which are generally made from gelatin blends optionally blended with plasticizers, as well as a starch capsule. In addition, dosage unit forms can contain various other materials that modify the physical form of the dosage unit, for example, coatings of sugar, shellac, or enteric agents. Other oral dosage forms syrup or elixir may contain sweetening agents, preservatives, dyes, colorings, and flavorings.

According to a further object, the present invention also concerns a formulation of the nanoparticles of the invention, said formulation comprising:
said nanoparticles in solution or suspension in water, in a concentration of 0.5 to 10 mg/ml equivalent of said chemotherapeutic agent, such as IRN, preferably 0.5 to 2 mg/ml, more preferably 1 to 1,5 mg/ml; and
said solution or suspension comprising 0.5 to 5%, advantageously 1%, of a cryoprotector agent, such as glucose, mannitol, lactose, more advantageously 1% of glucose.

The present invention also concerns the lyophilized nanoparticles comprising said chemotherapeutic agent of the invention.

Said lyophilized nanoparticles are particularly suitable for oral administration, for the treatment and/or prevention of cancer.

The present invention also concerns a process of lyophilizing the nanoparticles of the invention. Said process comprises the following steps:
Step 1: freezing the above formulation of the invention: Generally, suitable freezing temperatures are comprised between −80° C. and −30° C., preferably around −55° C. Freezing may be conducted during periods from 10 minutes to 10 days, generally during about 5 hours
Step 2: primary drying said freezed formulation. Primary drying temperatures may be comprised between +5° C. to +50° C., such as around +20° C., at reduced pressures, comprised between 50 and 200 μBar, advantageously around 120 μBar. Primary drying may be conducted from few minutes to few days, advantageously around 1 day.
Step 3: secondary drying said primary dried freezed formulation. Secondary drying may be achieved by one or more stage(s) of reducing the drying pressure and/or increasing the drying temperature. Preferable secondary drying conditions include one stage at a temperature around +20° C. at P=80 μBar, followed by a second stage at T=+35° C. and P=80 μBar. Secondary drying stage(s) be conducted from few minutes to few days, advantageously around 5 hours:

The obtained product allows immediate reconstitution of homogenous liquid formulation when stirred with water.

According to a still further object, the present invention also concerns a medicament comprising at least one nanoparticle according to the invention in a pharmaceutically acceptable vehicle, said medicament being for oral administration.

According to a still further object, the present invention also concerns a medicament comprising at least one nanoparticle according to the invention in a pharmaceutically acceptable vehicle for the treatment and/or the prevention of cancer, said treatment and/or prevention comprising the oral administration of said medicament.

According to a further object of the invention, the treatment may also include the administration of one or more further anticancer agent, such as, but not limited to, 5-fluorouracil or other antimetabolite fluoropyrimidine like capecitabine or Tegafur-Uracile (“UFT”).

According to a further object, the present invention also concerns the process of preparation of the nanoparticles of the invention, said process comprising the steps consisting in:
preparing a polymerisation medium comprising at least one acid, said cyclic oligosaccharide, such as cyclodextrin and optionally said tensioactive or surfactive agent such as poloxamer 188;
mixing said chemotherapeutic agent with the polymerisation medium;
mixing with the monomer of said polymer;
allowing polymerisation to occur.

### DESCRIPTION OF THE DRAWINGS

FIG. 1 illustrates the tumor evolution of treated mice where Relative Tumor Volume (RTVm) represents the tumor evolution related to the volume registered at the beginning of the treatment with a formulation of the invention.

FIG. 2 illustrates the orthotopic tumor volume evolution of treated mice assessed at two sacrifice time, when administered with a formulation of the invention.

FIG. 3 illustrates the weight evolution of treated mice where Relative Body Weight (RBWm) represents the body weight evolution related to the weight registered at the beginning of the experimentation per group, when administered with a formulation of the invention.

FIG. 4 illustrates the tumor evolution of treated mice where Relative Tumor Volume (RTVm) represents the tumor evolution related to the volume registered at the beginning of the treatment with a formulation of the invention.

### EXAMPLES

The following examples are given as a non limiting illustration of the present invention.

### Example 1

### Raw Materials, and Main Protocols

### Raw Materials:

Active Principles;
Irinotecan (provided by Interchemical/Haorui),
Ellipticine derivatives (2-(beta piperidino-2-ethyl)9-hydroxy-ellipticinium) (provided by Novasep),
Doxorubicin,
Paclitaxel.

Excipients;

| | Provider | Sample number |
|---|---|---|
| HP-β Cyclodextrin | Roquette | ND |
| Rameb Cyclodextrin | Cyclolab | CYL 22-47 |
| Monohydrated citric acid | Cooper | P51825/1 |
| Poloxamer 188 | ND | POL01-003 |
| Monorex ® | BioAlliance Pharma | YGZ 004-02 |

### Method for Preparing the Polymerization Medium (pH Comprised Between 2 and 3.5)

Preparation of citric acid 0.1 M

Addition of poloxamer 188 (concentration between 0,5 and 15%), under stirring and till complete dissolution occurs

Addition of Cyclodextrin (concentration between 0.1 and 70%)

Adjustment to the required pH

Filtration on 0.2 μm filters.

### Method for Preparing Irinotecan, Doxorubicin, Paclitaxel or Ellipticine Derivatives (2-(Beta Piperidino-2-Ethyl)9-Hydroxyellipticinium)-SRN (5 ml Batches)

In a 10 ml flask, add 5 ml of
Irinotecan, paclitaxel or ellipticine derivatives between 0.5 and 2 mg/ml in the polymerization medium
Doxorubicin between 0.5 and 10 mg/ml

Magnetic stirring

Add slowly 50 to 200 μl of Monorex® under stirring

Let polymerization occurs up to 24 h period of time, under magnetic stirring and room temperature

Filtration onto 2 μm filters

### Example 2

### Encapsulations Studies and Optimization: Incidence of Increasing Quantities of IRN

Protocol: Solutions of 0.5, 0.75, 1, 2 & 5 mg/ml of IRN in pH 3.5 polymerization medium were prepared and placed in 10 ml flasks (5 ml of solution per flask). Polymerization were launched by adding dropwise 50 μl of Monorex® under magnetic stirring. After 24 h, suspensions were collected and analyzed by HPLC before and after filtration on 2 μm filters, before and after centrifugation at 50.000 rpm, 30 min at 20° C.

Results: Increasing quantities of irinotecan in a fixed quantity of monomer, lead to decreasing values of encapsulation yields (data not shown). Best results are obtained with 0,5 to 1,5 mg/ml IRN solutions with 50 μl of Monorex® with encapsulation yields >95%.

### Example 3

### IRN-SRN Preliminary in Vitro & in Vivo Results

### 3.1. In Vitro Cytotoxicity Results of IRN-SRN on HT29 Cells

The effect of the irinotecan-SRN was tested. SRN alone, irinotecan-SRN (with the weight ratio irinotecan/polymer equal to 1/20) as well as irinotecan alone were put in contact with growing HT29 cells for 4 days and the IC₅₀ were determined. Three different runs were performed and IC₅₀ (inhibition concentration 50%) was estimated.

The different efficacies determined are listed below:

TABLE 1
In vitro antitumor activity of the three test articles (IC50 values)

| | | | Experiment number | | | | | | | |
|---|---|---|---|---|---|---|---|---|---|---|
| | | | GF411 | | GF464 | | GF522 | | Mean IC 50 | |
| | Test | Inhib. | [IRN] | [Polymer] | [IRN] | [Polymer] | [IRN] | [Polymer] | [IRN] | [Polymer] |
| | article | Conc. | mg/mL | mg/mL | mg/mL | mg/mL | mg/mL | mg/mL | mg/mL | mg/mL |
| 1 | IRN- | IC50 | 0.80 | 15.99 | 0.83 | 16.66 | 1.14 | 22.75 | 0.82 | 18.47 |
| | SRN | | | | | | | | | |
| 2 | IRN | IC50 | 0.75 | | 0.80 | | 0.85 | | 0.80 | |
| | alone | | | | | | | | | |
| 3 | SRN | IC50 | | 33.86 | | 36.57 | | 34.94 | | 35.1 |
| | alone | | | | | | | | | |

In vitro, irinotecan-SRN exhibited a similar anticancer activity as irinotecan alone in the human colon cancer cell line HT-29, suggesting that entrapment of Irinotecan into the nanoparticles did not reduce its activity. One should however keep in mind that SN38 is the active metabolite of IRN and in order to be active it has to be metabolized by carboxylesterase enzymes. So this could be a limitation of the in vitro tests on cells.

It should be notice that blank nanoparticles (SRN alone) displayed a cytotoxic activity in vitro. Indeed, it has been described that the degradation of blank nanoparticles leads to polycyanoacrylate acid, which is known to be cytotoxic in vitro.

This in vitro study on the human colon cancer cell line HT-29 confirms the fact that irinotecan encapsulated in nanoparticles kept its efficacy and was not modified by the polymerisation process.

3.2. In Vivo Studies of IRN-SRN on Xenografted Mice

3.2.1. Single Maximum Tolerated Dose (MTD) Studies:

Oral Administration of IRN-SRN

Single dose MTD evaluation studies were first conducted. Three dose escalations were orally tested, 50 mg/kg; 100 mg/kg and 200 mg/kg. Each dose was tested on a group of 3 animals and the animals were monitored for 2 weeks, clinical toxicity signs were observed and the animals weight taken every two days.

At 50 mg/kg and 100 mg/kg orally, no signs of toxicity appeared and no body weight loss was observed. At 200 mg/kg, after 4 days two of the animals are at 94% and one is at 91.5% of their initial body weight but there were no apparent clinical toxicity signs. A reference group was dosed orally with CAMPTO® (IRN in solution) and showed no weight loss or clinical toxicity signs.

The Maximum Tolerated dose for oral dosage of IRN-SRN was 200 mg/kg in this experimental model.

3.2.2. MTTD (Maximum Total Tolerated Doses) Studies for Oral Administration of IRN-SRN

The schedule of the MTTD studies was one administration per week during three weeks. Each dose was tested on a group of 3 animals and the animals were monitored for 2 weeks after the end of the last treatment, clinical toxicity signs were observed and the animals weight taken every two days.

MTTD Studies Planned had the Following Schedule:

TABLE 2
Schedule of MTTD studies and clinical manifestations

| | | | Dose level | | |
|---|---|---|---|---|---|
| | Treatment | | [mg/kg BW] | Schedule | Clinical symptoms |
| 1 | Vehicle | iv | 10 | J = 0, 7, 14 | No |
| 2 | IRN-SRN | po | 200 | J = 0, 7, 14 | No clinical sign |
| 3 | IRN | iv | 25 | J = 0, 7, 14 | No clinical sign |
| 4 | IRN | po | 200 | J = 0, 7, 14 | No clinical sign |

For oral administration, the MTTD could not be determined due to the highest volume of dosing required. Therefore, a maximum feasible dose has been determined at 200 mg/kg in this experimental model.

3.3. Oral MTTD Studies on Xenografted Mice

A tolerance and efficacy study on xenografted mice, in order to mimic as close as possible the in vivo model of colonic tumor, was carried out. As xenografted mice are very sensitive, the MTTD and efficacy study was done on mice with a subcutaneously implanted HT-29 colorectal human tumor cells.

The schedule was the following:

TABLE 3
Schedule of MTTD studies and clinical manifestations

| | | | Dose level | | |
|---|---|---|---|---|---|
| | Treatment | | [mg/kg BW] | Schedule | Clinical symptoms |
| 1 | Vehicle | po | 10 ml/kg | J = 1 to 5 | No |
| | | | BW | | |
| 2 | IRN-SRN | po | 200 | J = 1 to 5 | No clinical signs |
| 3 | IRN-SRN | po | 100 | J = 1 to 5 | No clinical signs |
| 4 | IRN-SRN | po | 50 | J = 1 to 5 | No clinical signs |
| 5 | IRN | po | 200 | J = 1 to 5 | Important weight loss |
| | | | | | (>10%), treatment was |
| | | | | | interrupted |
| 6 | IRN | po | 100 | J = 1 to 5 | Weight loss, but mice |
| | | | | | recovered the week after |

IRN-SRN administered orally every day for 5 days showed no clinical signs of toxicity (table 3). On the other hand, IRN induced an important weight loss.

As for the previous experiment with an administration schedule of three oral administrations every week for 3 weeks, the MTTD could not be determined due to the highest volume of dosing required. Therefore, a maximum feasible dose has been determined at 200 mg/kg in this experimental model.

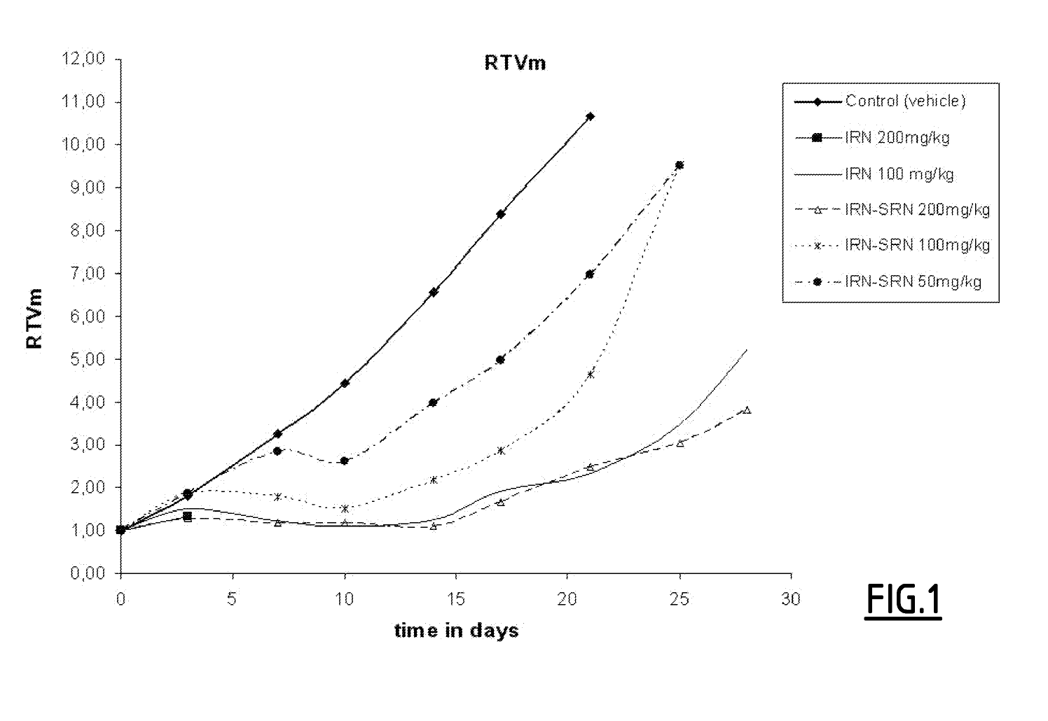

An examination of the tumor evolution was also considered during this experiment. Treatment with IRN-SRN (50, 100, and 200 mg/kg) induces a significant reduction of the tumor volumes (see FIG. 1).

At this step, IRN-SRN have shown interesting properties. Indeed, they not only have a dose dependent efficacy and an antitumor effect comparable to CAMPTO®, but they are also better tolerated.

3.4. Efficacy Study of IRN-SRN Against HT29 Tumor Xenografts Growing Orthotopically

The objective is to test the efficacy of IRN-SRN administered per os on HT-29 colorectal tumors growing orthotopically. Dosage and schedule used were chosen from results of tolerance study.

Orthotopic grafts: tumor fragments about 30 mm³ are sutured against the caecum wall as described above. Groups of 15 mice are randomly affected to the treatments 2 weeks post-grafting.

The treatment were adjusted to lower doses if the animals loose weight (>10% for 72 consecutive hours). In each group, a pool of 7-8 mice were sacrificed at day 19 and day 45 after start of treatment. Tumor samples were fixed in 10% formol and processed for histological examination.

| Group | Treatment | Dosage, schedule | Nb of mice |
|---|---|---|---|
| 1 | Control | vehicle, po, qd×5 | 15 |
| 2 | IRN | 100 mg/kg, po, qd×5 | 15 |
| 5 | IRN-SRN | 200 mg/kg, po, qd×5 | 15 |

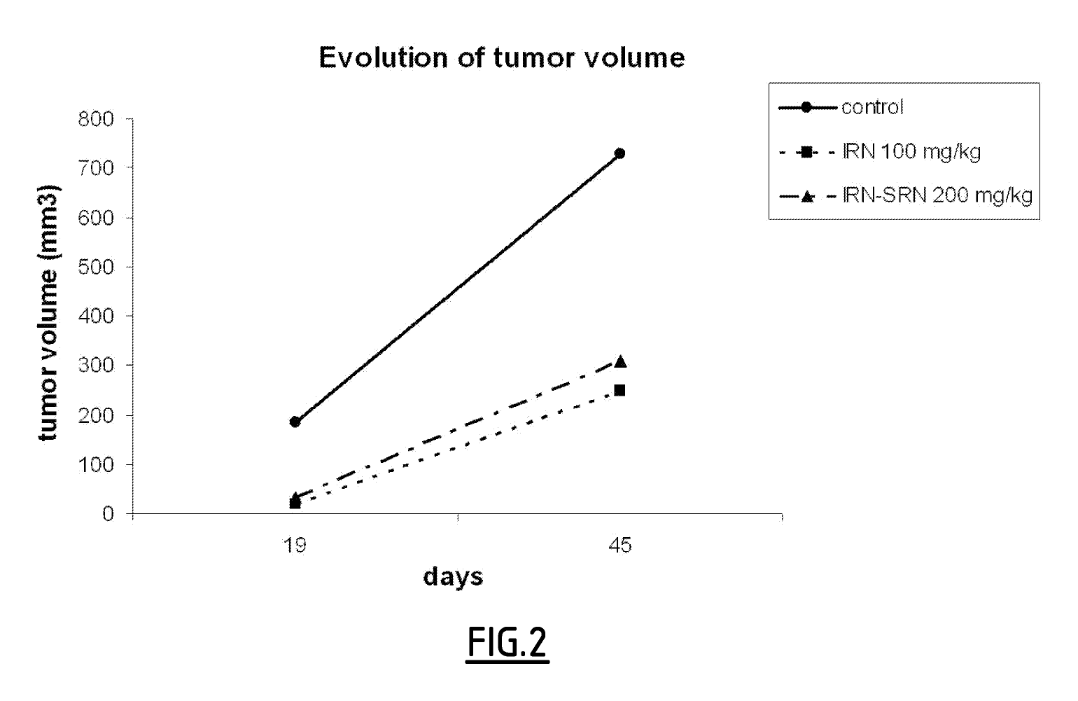

Treatment with IRN-SRN (200 mg/kg) induces a significant reduction of the tumor volumes (see FIG. 2).

### Example 4

### In vivo Anti-Tumor Effect of IRN-SRN and Irinotecan in the HT29 Xenograft Model of Human Colorectal Adenocarcinoma

4.1. Material & Methods

### Identification of Animals and Formulation of Dosage Groups

All animals are weighed before each experiment and are subdivided into the different dosage groups.

Each cage is identified by a paper tag indicating: experiment code, cage number, mice number, tumor code, name of the test item, dose and route of administration.

### Tumor Induction

The HT29 colorectal tumor cell line has been established as a subcutaneously growing tumor xenograft into nude mice.

Tumor xenografts are maintained by serial transplantation into immunodeficient mice. Mice received subcutaneous grafts of tumor fragments originated from a previous passage. Fragments for this assay will originate from 5 donor mice bearing the previous tumor passage and sacrificed when the tumor reached 12 to 15 mm of diameter. All mice from the same experiment were implanted on the same day. It was planned to include at least 10 mice per group.

Tumor-bearing donor mice were sacrificed by cervical dislocation. The tumor was aseptically excised. Tumors were deposited in a Petri dish containing culture medium and dissected carefully to remove the fibrous capsule usually surrounding the tumor. Necrotic tumors were rejected. Tumor tissue was maintained in culture medium during the transplantation procedure. One tumor led up to 8 transplants, each fragment measuring approximately 40 mm³.

Subcutaneous implantations were performed aseptically. After anaesthesia with ketamine/xylazine, and sterilisation of the skin with a betadine solution, the skin was incised at the level of the interscapular region, and a fragment of tumor was placed in the subcutaneous tissue. Skin was closed with clips.

### Inclusion Criteriae and Randomization

Only healthy mice aged 7 to 9 weeks and weighing at least 20 g were included in the study. At the step of transplantation, tumor fragments were randomly distributed onto nude mice and were be individually identified by a number and allocated to a tumor fragment. Treatments were randomly attributed to boxes housing 3 to 5 mice.

### Chemotherapy Protocol

Drug Administration

IRN-SRN and IRN were administered by oral or intravenous route following the indicated regimens. Mice received variant volumes of IRN-SRN in order to obtain the wanted doses (about 125 to 750 μL). Irinotecan was diluted in order to administrate 500 μL by oral route.

Vehicle

Control animals received the vehicle used to prepare the IRN-SRN solution (polymerization medium).

### Experimental Protocols

Tolerance Study (Step 1)

The objectives were to test the tolerance to Irinotecan and IRN-SRN given either iv or per os to nude mice bearing HT29 tumors, according to the defined dosage and administration route.

When subcutaneous HT29 xenografts are detectable and reach a mean tumor volume of 150 mm3, groups of mice are randomly affected to the treatments. Mice without tumors were eliminated.

The treatment were adjusted to lower doses if the animals loose weight (>15% for 72 consecutive hours).

Mice in the control group received vehicle under a 750 μl volume.

| Group | Treatment | Dosage, schedule | Nb of mice |
|---|---|---|---|
| 1 | Control Vmax | Vehicle, po, qd×5 | 5 |
| 2 | IRN | 20 mg/kg, iv, (qwk)×2 | 5 |
| 3 | IRN | 100 mg/kg, po, qd×5 | 5 |
| 4 | IRN-SRN | 50 mg/kg, po, qd×5 | 5 |
| 5 | IRN-SRN | 100 mg/kg, po, qd×5 | 5 |
| 6 | IRN-SRN | 200 mg/kg, po, qd×5 | 5 |
| 7 | IRN-SRN | 300 mg/kg, po, qd×5 | 5 |

Efficacy Study of IRN-SRN Against HT29 Tumor Xenografts Growing Subcutaneously (Step 2)

The objectives were to test the efficacy of IRN-SRN administered per os on HT-29 colorectal tumors growing subcutaneously. Dosage and schedule used were chosen from results of tolerance study.

Groups of 10 mice were randomly affected to the treatment groups to give identical mean tumor volumes between groups when tumor volume is between 60-150 mm³. Treatments started on the next day following group affectation (Day 1).

The treatment will be adjusted to lower doses if the animals loose weight (>15% for 72 consecutive hours).

| Group | Treatment | Dosage, schedule | Nb of mice |
|---|---|---|---|
| 1 | Control | vehicle, po, qd×5 | 10 |
| 2 | IRN | 100 mg/kg, po, qd×5 | 10 |
| 3 | IRN | 20 mg/kg, iv, (qwk)×2 | 10 |
| 4 | IRN-SRN | 300 mg/kg, po, qd×5 | 10 |
| 5 | IRN-SRN | 100 mg/kg, po, qd×5 | 10 |
| 6 | IRN-SRN | 33 mg/kg, po, qd×5 | 10 |
| 7 | SRN-Control | po, qd×5 | 6 |

Bio-Distribution Study of IRN-SRN (Step 3)

Blood was sampled by cardiac puncture on xylamine-ketamine-anesthesized mice 5 min, 10 min, 15 min, 30 min, 60 min, 2 h, 4 h, 8 h, 16 h, 30 h, and 48 h after a single iv injection, or 15 min, 30 min, 60 min, 2 h, 4 h, 8 h, 16 h, 30 h, and 48 h after a single po administration.

Tumors were dissected and flash-frozen 60 min, 4 h, 8 h, 16 h, 30 h, and 48 h after a single iv injection or po administration.

Three mice were used per time-point. Only one sampling was performed on the control group.

| Group | Treatment | Dosage, schedule | Nb of mice |
|---|---|---|---|
| 1 | Control | vehicle, po | 3 |
| 2 | IRN | 100 mg/kg, po | 27 |
| 3 | IRN | 20 mg/kg, iv | 33 |
| 4 | IRN-SRN | 300 mg/kg, po | 27 |
| 6 | IRN-SRN | 100 mg/kg po | 27 |

Plasma samples were prepared for CPT11 and its active metabolite SN38 assay.

### Tumor Growth

To evaluate the antitumor activity of drugs on human xenografts, tumor volumes are evaluated by measuring biweekly tumor diameters with a calliper. The formula TV (mm³)=[length (mm)×width (mm)2]/2 is used, where the length and the width were the longest and the shortest diameters of each tumor, respectively.

Relative tumor volume (RTV): is calculated as the ratio of the volume at the time t divided by the initial volume at day 1 and multiplied by 100. Curves of mean RTV as a function of time in treated and control groups are generated and presented in the report.

### Toxicity Parameters

Toxicity of the different treatments are determined as: body weight loss percent (% BWL max)=100−(mean BWx/mean BW1x 100), where BWx is the mean BW at the day of maximal loss during the treatment and BW1 is the mean BW on the 1st day of treatment.

Lethal toxicity is any death in treated group.

### Clinical Observations

Mortality

Animals are inspected every day for mortality.

Clinical Signs

Mice are observed daily for physical appearance, behaviour and clinical changes.

Clinical observations are made in order to detect abnormalities related to the involvement of tegumental, digestive, musculoskeletal, respiratory, genitourinary apparatus and central nervous system.

All signs of illness, together with any behavioural change or reaction to treatment, are recorded for each animal. All clinical signs are recorded for individual animal, in the laboratory notebook throughout the whole study.

Body Weight

All animals are weighted during the whole treatment period, in order to adjust the volume of drug administration and to calculate the percent body weight loss due to the different treatments.

4.2. Results

Tolerance Study (Step 1)

TABLE 4
Relative body weight (RBW) as a function of time

| | Control | | IRN | | IRN | |
|---|---|---|---|---|---|---|
| | p.o. | | 100 mg/kg p.o | | 20 mg/kg i.v. | |
| Day | RBW m | SD | RBW m | SD | RBW m | SD |
| 0 | 1.00 | 0.00 | 1.00 | 0.00 | 1.00 | 0.00 |
| 1 | 1.01 | 0.01 | 0.98 | 0.01 | 1.00 | 0.01 |
| 2 | 0.99 | 0.01 | 0.93 | 0.01 | 0.95 | 0.01 |
| 3 | 1.03 | 0.01 | 0.90 | 0.01 | 0.95 | 0.01 |
| 4 | 1.01 | 0.01 | 0.88 | 0.01 | 0.96 | 0.01 |
| 7 | 1.00 | 0.01 | 0.83 | 0.03 | 0.96 | 0.02 |
| 10 | 0.97 | 0.02 | 0.90 | 0.03 | 0.95 | 0.02 |
| 15 | 0.99 | 0.01 | 1.00 | 0.02 | 0.97 | 0.02 |
| | Control | | IRN-SRN | | IRN-SRN | |
| | p.o. | | 50 mg/kg p.o. | | 100 mg/kg p.o. | |
| Day | RBW m | SD | RBW m | SD | RBW m | SD |
| 0 | 1.00 | 0.00 | 1 | 0 | 1 | 0 |
| 1 | 1.01 | 0.01 | 1.01 | 0.01 | 1.00 | 0.00 |
| 2 | 0.99 | 0.01 | 0.98 | 0.01 | 0.97 | 0.01 |
| 3 | 1.03 | 0.01 | 0.97 | 0.02 | 0.98 | 0.01 |
| 4 | 1.01 | 0.01 | 1.00 | 0.01 | 0.97 | 0.01 |
| 7 | 1.00 | 0.01 | 0.98 | 0.01 | 0.97 | 0.01 |
| 10 | 0.97 | 0.02 | 0.97 | 0.02 | 0.96 | 0.01 |
| 15 | 0.99 | 0.01 | 0.98 | 0.04 | 1.03 | 0.02 |

| | Control | | IRN-SRN | | IRN-SRN | |
|---|---|---|---|---|---|---|
| | p.o. | | 200 mg/kg p.o. | | 300 mg/kg p.o. | |
| Day | RBW m | SD | RBW m | SD | RBW m | SD |
| 0 | 1.00 | 0.00 | 1 | 0 | 1 | 0 |
| 1 | 1.01 | 0.01 | 0.98 | 0.004 | 0.984 | 0.007 |
| 2 | 0.99 | 0.01 | 0.96 | 0.005 | 0.948 | 0.006 |
| 3 | 1.03 | 0.01 | 0.95 | 0.012 | 0.968 | 0.009 |
| 4 | 1.01 | 0.01 | 0.95 | 0.014 | 0.984 | 0.012 |
| 7 | 1.00 | 0.01 | 0.96 | 0.008 | 0.9723 | 0.018 |
| 10 | 0.97 | 0.02 | 0.97 | 0.0156 | 0.961 | 0.026 |
| 15 | 0.99 | 0.01 | 1.04 | 0.0128 | 1.038 | 0.017 |

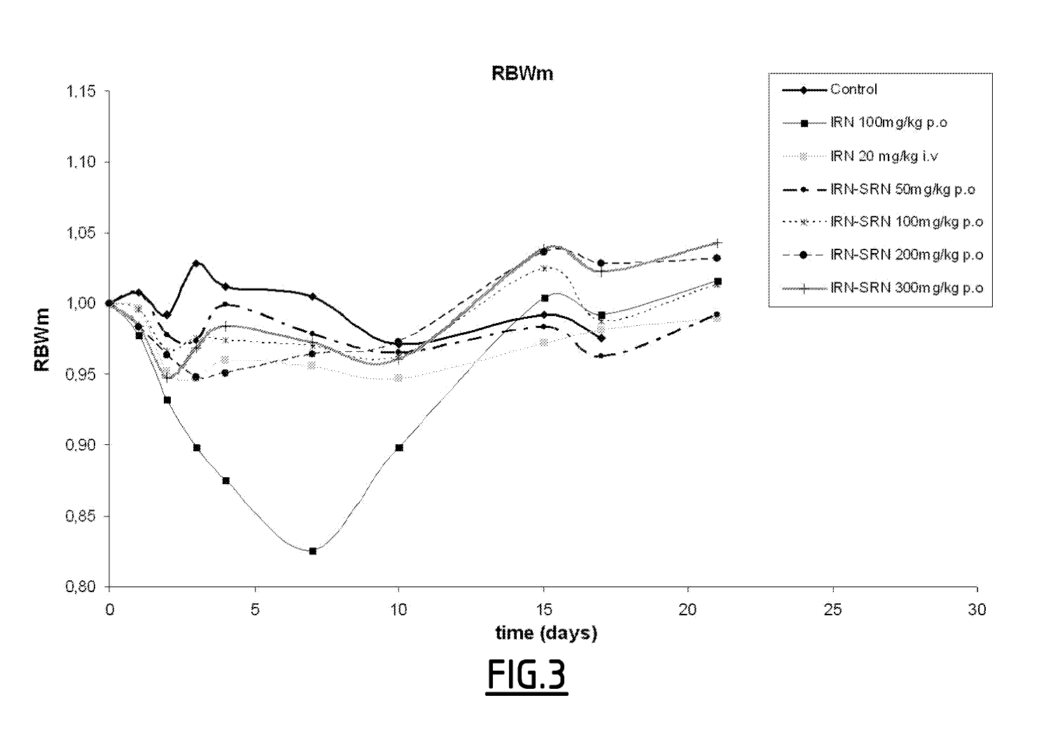

No body weight loss was observed in the IRN-SRN groups (50, 100, 200 and 300 mg/kg) after oral administration whereas more than 15% body weight loss was observed in the oral IRN alone group (see FIG. 3). It indicated that IRN-SRN is better tolerated than IRN alone after oral administration. It confirms that IRN-SRN could be able to reduce side effects of oral administration of irinotecan.

Efficacy Study of IRN-SRN Against HT29 Tumor Xenografts Growing Subcutaneously (Step 2)

TABLE 5
Relative tumor volume (RTV) as a function of time

| | Control | | IRN | | IRN | |
|---|---|---|---|---|---|---|
| | p.o. | | 100 mg/kg p.o. | | 20 mg/kg i.v | |
| Day | RTV m | SD | RTV m | SD | RTV m | SD |
| 0 | 1 | 0 | 1 | 0 | 1 | 0 |
| 3 | 3.16 | 0.68 | 2.20 | 0.36 | 2.02 | 0.26 |
| 7 | 4.90 | 0.72 | 1.74 | 0.26 | 2.26 | 0.43 |
| 10 | 7.01 | 1.31 | 1.30 | 0.13 | 2.90 | 0.60 |
| 15 | 12.35 | 2.31 | 1.98 | 0.22 | 4.71 | 1.07 |
| | Control | | IRN-SRN | | IRN-SRN | |
| | p.o. | | 50 mg/kg p.o. | | 100 mg/kg p.o. | |
| Day | RTV m | SD | RTV m | SD | RTV m | SD |
| 0 | 1 | 0 | 1 | 0 | 1 | 0 |
| 3 | 3.16 | 0.68 | 2.48 | 0.33 | 2.59 | 0.38 |
| 7 | 4.90 | 0.72 | 2.59 | 0.47 | 2.70 | 0.24 |
| 10 | 7.01 | 1.31 | 3.89 | 0.89 | 2.17 | 0.29 |
| 15 | 12.35 | 2.31 | 7.69 | 2.88 | 4.13 | 0.62 |
| | Control | | IRN-SRN | | IRN-SRN | |
| | p.o | | 200 mg/kg p.o. | | 300 mg/kg p.o. | |
| Day | RTV m | SD | RTV m | SD | RTV m | SD |
| 0 | 1 | 0 | 1 | 0 | 1 | 0 |
| 3 | 3.16 | 0.68 | 2.40 | 0.60 | 1.67 | 0.19 |
| 7 | 4.90 | 0.72 | 1.70 | 0.31 | 1.80 | 0.21 |
| 10 | 7.01 | 1.31 | 1.30 | 0.26 | 1.31 | 0.15 |
| 15 | 12.35 | 2.31 | 2.25 | 0.42 | 1.57 | 0.44 |

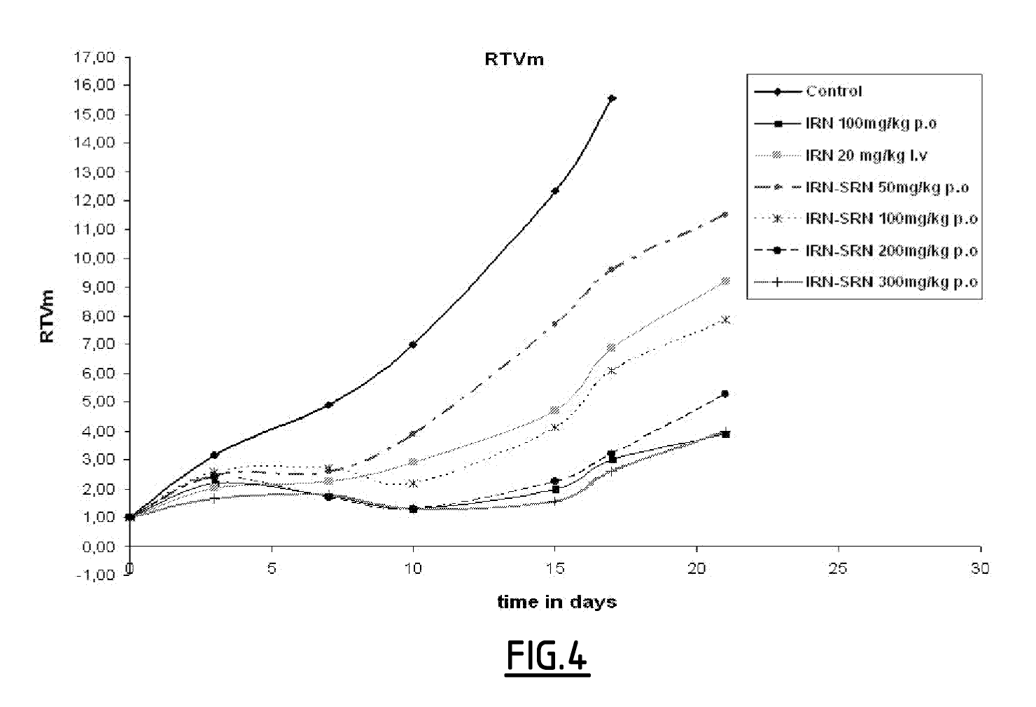

Treatment with IRN-SRN (50, 100, 200 and 300 mg/kg) also induces a significant reduction of the tumor volumes (see FIG. 4). Moreover administration of different doses show a dose related effect of IRN-SRN. Indeed efficacy of the treatment increases with the administrated dose of IRN-SRN. Oral IRN-SRN at 200 and 300 mg/kg is as efficient as oral IRN alone at 100 mg/kg and is better tolerated.

Bio-Distribution Study of IRN-SRN (Step 3)

TABLE 6
Pharmacokinetics parameters of IRN and of IRN-SRN.

| | IRN | IRN | IRN-SRN | IRN-SRN |
|---|---|---|---|---|
| | 20 mg/ | 100 mg/ | 100 mg/ | 300 mg/ |
| IRINOTECAN | Kg iv | Kg po | Kg po | Kg po |
| AUC | 1284 | 1741 | 1632 | 9698 |
| (ng h/mL) | | | | |
| Clearance | 15.6 | 57.4 | 61.2 | 30.9 |
| (mL/h) | | | | |
| T½ | 1.22 | 1.19 | 3.93 | 4.68 |
| (h) | | | | |
| Cmax | | 477 | 171 | 1516 |
| (ng/mL) | | | | |
| Tmax | | 2.06 | 4.35 | 0.85 |
| (h) | | | | |
| F % | | 27.1 | 24.9 | 50.3 |

AUC=Area Under Curve corresponds to the integral of the plasma concentration over a given period of time.

Clearance=Coefficient representing the ability of an organ or tissue to eliminate a given substance from a fluid of the organism. The term normally used is “renal clearance”, i.e. the ratio of the urinary flow of a body and its concentration in the plasma. Clearance shows how the medication is eliminated.

T½=The plasma half-life of a drug (T½) is the time required for the plasma concentration to diminish by half, for example, from 100 to 50 mg/L. Knowing the half-life makes it possible to plan the frequency of drug administration (number of daily doses) to obtain the desired plasma concentration. In the huge majority of cases, half-life is independent of the dose of medication administered. In some exceptional cases, it varies with the dose: it may increase or decrease according to the occurrence of saturation of a mechanism (elimination, catabolism, adherence to plasma proteins, etc.).

Cmax=Maximum plasma concentration. The term half maximal effective concentration (EC50) refers to the concentration of a drug, antibody or toxicant which induces a response halfway between the baseline and maximum after some specified exposure time. It is commonly used as a measure of drug potency and toxicity.

Tmax=Time to attaining Cmax (correlation between Cmax and time)

F%=Bioavailability indicates the percentage of administered medication that reaches the central compartment. It is generally measured by comparing the AUCs obtained after administration of the same medication intravenously and by another route, usually oral. After intravenous administration, the AUC obtained corresponds to a bioavailability that, by definition, is 100%; following buccal administration, the AUC corresponds to an identical bioavailability in the ideal case, but generally corresponding to lower or occasionally nil bioavailability.

The pharmacokinetic results of irinotecan after administration of oral IRN-SRN at 300 mg/kg and iv free IRN solution at 20 mg/kg were as followed: AUC of 9698 ng.h/ml vs 1284 ng.h/ml, Clearance of 30.9 l/h versus 15.6 l/h, T½ of 4.68 h versus 1.22 h respectively The relative bioavailability of irinotecan obtained with IRN-SRN at 300 mg/kg was 50%.

TABLE 7
Pharmacokinetics parameters of SN-38.

| | IRN | IRN | IRN-SRN | IRN-SRN |
|---|---|---|---|---|
| | 20 mg/ | 100 mg/ | 100 mg/ | 300 mg/ |
| SN-38 | Kg iv | Kg po | Kg po | Kg po |
| AUC | 6403 | 11291 | 6793 | 21056 |
| (ng h/mL) | | | | |
| T½ | 1.52 | 1.42 | 4.32 | 3.7 |
| (h) | | | | |
| Cmax | | 2609 | 629 | 2516 |
| (ng/mL) | | | | |
| Tmax | | 2.06 | 2.28 | 2.17 |
| (h) | | | | |
| F % | | 3.53 | 21.2 | 21.9 |

For SN-38 (the active metabolite of irinotecan), the AUC was 21056 ng.h/ml versus 6403 ng.h/ml, the T½ was 3.7 h versus 1.52 h respectively after administration of oral IRN-SRN at 300 mg/kg and iv free IRN solution at 20 mg/kg. The relative bioavailability of SN-38 obtained with IRN-SRN at 300 mg/kg was 22%.

The pharmacokinetic study demonstrates an improved bioavailability, and moreover, a significant prolonged half life with this IRN-SRN oral formulation compared to the free intravenous or oral IRN solution.

### Example 5

### Freeze Drying of IRN-SRN Formulation and Reconstitution Tests

5.1. Introduction

This study had been performed to determine an optimal formulation of IRN-SRN for the freeze-drying process.

Reconstitution tests had been performed after lyophilisation to determine the condition of reconstitution for each formulation.

To determine the optimal formulation a granulometric control had been performed on each freeze-dried product.

Tests have been made on the following formulation:
1 vial: IRN-SRN in water (1 mg/ml equivalent IRN) without cryoprotector
1 vial: IRN-SRN in water (1 mg/ml equivalent IRN) 1% glucose
1 vial: IRN-SRN in water (1 mg/ml equivalent IRN) 1% mannitol
1 vial: IRN-SRN in water (1 mg/ml equivalent IRN) 5% glucose, 0.5% lactose
1 vial: IRN-SRN in water (1.5 mg/ml equivalent IRN) without cryoprotector
1 vial: IRN-SRN in water (1.5 mg/ml equivalent IRN) 1% glucose
1 vial: IRN-SRN in water (1.5 mg/ml equivalent IRN) 1% mannitol
1 vial: IRN-SRN in water (1.5 mg/ml equivalent IRN) 5% glucose, 0.5% lactose

5.2. Freeze Dried Process and Results

Conditions of freezing were: Temperature of shelf=−55° C., duration 4 h30

Conditions of primary drying: Temperature of shelf=+20° C., P=120 μBar, observed duration 19 h

Conditions of secondary drying:

i. Temperature of shelf=+20° C., P=80 μBar, duration=5 h30

ii. Temperature of shelf=+35° C., P=80 μBar, duration=4 h00

The total duration of the freeze-drying process was around 33 hours.

After the freeze-dried process, the visual appearance of vials is variable:
Formulation without cryoprotector: appearance is conform
Formulation with 1% glucose: appearance is conform
Formulation with 1% mannitol: appearance is conform, but the product sticks to the vials
Formulation with 5% glucose and 0.5% lactose: appearance is not conform due to the vitrification of glucose during the freezing step. A retraction of the cake is observed.

5.3. Protocol of Reconstitution and Results

Vials are removed from the fridge and placed at room temperature for at least 30 minutes

A first needle is placed through the stopper to allow the air to enter during the addition of water

The appropriate amount of water is added in each vial on the cake
Reconstitution test with 5 ml: add 1 ml, and after the complete dissolution of cake add 4 ml more in the vial
Reconstitution with 250 μl: add directly on the cake 250 μl of water

After having added water on the cake vials are checked vigorously and manually. In the case of the presence of aggregates in the vial a “vortex” is used.

### Summary of Reconstitution Test in 5 ml

| Formulation | | Appearance | Reconstitution |
|---|---|---|---|
| 1 mg/ml | Without | Strong yellow, | Need to vortex |
| | cryoprotector | conform | sample. |
| | 1% glucose | Strong yellow, | Immediate |
| | | conform | reconstitution |
| | 1% mannitol | Yellow, conform | Immediate |
| | | but sticks on the | reconstitution |
| | | vial | |
| | 5% glucose, 0.5% | Strong yellow, | Immediate |
| | lactose | retracted cake | reconstitution |
| 1.5 mg/ml | Without | Pale yellow, | Not reconstituted in |
| | cryoprotector | conform | 1 ml, need to vortex. |
| | | | Reconstitution ok |
| | | | with 4 ml more. |
| | 1% glucose | Pale yellow, | Immediate |
| | | conform | reconstitution |
| | 1% mannitol | Pale yellow, | Immediate |
| | | conform | reconstitution |
| | 5% glucose, 0.5% | Pale yellow, | Immediate |
| | lactose | retracted cake | reconstitution |

The most difficult formulation to reconstitute is the one which does not contain any cryoprotector. The reconstitution is satisfying for the other formulations.

### Summary of Reconstitution Test in 250 μl

| Formulation | | Appearance | Reconstitution |
|---|---|---|---|
| 1 mg/ml | Without | Strong yellow, | In 5 ml need to |
| | cryoprotector | conform | vortex >3 min |
| | 1% glucose | Strong yellow, | Immediate |
| | | conform | reconstitution, |
| | | | vortex for last |
| | | | aggregates |
| | 1% mannitol | Yellow, conform | Need to vortex, |
| | | but sticks on the | aggregates remain |
| | | vial | and product sticks |
| | | | on the vial |
| | 5% glucose, 0.5% | Strong yellow, | Immediate |
| | lactose | retracted cake | reconstitution |
| 1.5 mg/ml | Without | Pale yellow, | Need to vortex, |
| | cryoprotector | conform | aggregates remain |
| | 1% glucose | Pale yellow, | Immediate |
| | | conform | reconstitution |
| | 1% mannitol | Pale yellow, | Immediate |
| | | conform but sticks | reconstitution |
| | | on the vial | |
| | 5% glucose, 0.5% | Pale yellow, | Immediate |
| | lactose | retracted cake | reconstitution |

The most difficult formulation to reconstitute is the one which does not contain any cryoprotector. Even after using vortex to mix the sample many aggregates remain. The formulation containing 1% of mannitol the product sticks on the vial. The reconstitution is saisfying for the other formulations.

In conclusion, preferred formulations for lyophilisation contain 1% glucose. The product can be reconstituted easily either in 5 ml of water or 250 μl of water, and the granulometric profile is conform for both formulation (1 mg/ml and 1.5 mg/ml).

### Example 6

### Measure of Entrapped Cyclodextrins in IRN-SRN

6.1. Raw Materials, and Main Protocols

6.1.1. Raw Materials

| | Provider |
|---|---|
| HP-β Cyclodextrin | Roquette |
| Monohydrated citric acid | Cooper |
| Poloxamer 188 | BASF |
| Phenolphtalein 1% in ethanol | Sigma |
| Ethanol | Carlo Erba |
| Sodium carbonate | Sigma |
| Irinotecan | Antibioticos |
| Monorex ® | BioAlliance Pharma |

In alkaline solution, Phenolphtalein is pink coloured (anionic form of phenolphthalein). This anionic form has a characteristic absorption peak at 554 nm. When phenolphtalein is added to a solution containing HP-bCD, some of the PP anions form inclusion complexes with the HP-b-CD and become colourless. It induces a reduction in the intensity of the absorption peak at 554 nm.

A calibration has been done using different polymerization medium including HP-bCD concentrations from 0 to 0.15%.

The method has been validated with a 1 mg/mL irinotecan solution in a polymerization medium containing 0.1% HP-bCD.

Cyclodextrins amounts inside the nanoparticles were determined by indirect dosage: the nanoparticle suspensions were centrifuged at 50000 rpm during 30 minutes at 20° C. The supernatant was analyzed and the HP-bCD concentration entrapped into the nanoparticles is determined by difference.

6.1.2. Main Protocol

Method for Preparing a Polymerization Medium Containing Known Concentrations HP-bCD for Calibration (pH Comprises Between 2 and 3.5)
Preparation of citric acid 0.1M
Addition of poloxamer 188 (concentration between 0,5 and 1%) in the previous solution, under stirring and till complete dissolution occurs
Addition of Cyclodextrin (concentration between 0 and 0.15%)
Adjustment to the required pH

Method for Preparing the Samples for Analysis

The following solutions were prepared:
8% of sample (Irinotecan in polymerization medium and supernatant),
10% of a 0.01% phenolphthalein solution in ethanol,
10% of a 0.04 mol.L-1 sodium carbonate solution in water
72% water

The obtained mixtures were stirred until equilibrium (at least 48 h).

### Analysis

The absorbance was scanned from 200 nm to 800 nm to determine the formation of an inclusion complex and the characteristic absorption wavelength. At the characteristic absorption wavelength (554 nm), absorbance was measured to determine the amount of PP in solutions since the complexed form is colourless.

6.2. Results

The absorbance was calibrated depending on the % HP-bCD.

### Before Freeze Drying:

### First Experiment

| | % HP-bCD | | % HP-bCD entrapped |
|---|---|---|---|
| Sample | measured | SD | into the nanoparticles |
| Control: | 0.094% | 0.003% | |
| IRN 1 mg/mL | | | |
| 0.1% HP-bCD | | | |
| Supernatant | 0.022% | 0.003% | 78% |
| NP 1 mg/ml | | | |
| Supernatant | 0.038% | 0.014% | 75% |
| NP 1.5 mg/ml | | | |

### Second Experiment

| | % HP-bCD | | % HP-bCD entrapped |
|---|---|---|---|
| Sample | measured | SD | into the nanoparticles |
| Control: | 0.109% | 0.005% | |
| IRN 1 mg/mL | | | |
| 0.1% HP-bCD | | | |
| Supernatant | 0.023% | 0.005% | 77% |
| NP 1 mg/ml | | | |
| Supernatant | 0.023% | 0.002% | 77% |
| NP 1 mg/ml | | | |

### After Freeze-Drying

| | % HP-bCD | | % HP-bCD entrapped |
|---|---|---|---|
| Sample | measured | SD | into the nanoparticles |
| Supernatant | 0.015% | 0.003% | 85% |
| NP 1 mg/ml | | | |

### Example 7

### Doxorubicin-SRN Administered by Oral Route

7.1. Materials and Methods

Animals

Female 8 week-old C57BL/6J mice were provided by Harlan (Gannat, France). Mice were maintained for acclimatization for 7 days before oral administration.

The study involved 21 female C57BL/6J mice.

Drug Administration

Treatments have been Carried out as Follow:

| | | | | | Quantity of | |
|---|---|---|---|---|---|---|
| | Number of | | | Doxorubicine | Doxorubicin | |
| | animals per | | | Dose | administered (mg, | Treatment |
| Group | group | Treatment | Route | (mg/kg) | mouse weight = 20 g) | schedule |
| 1 | 3 | Doxorubicin- | oral | 10 | 0.2 | Q1D×1 |
| | | SRN | | | | |
| 2 | 3 | Doxorubicin- | oral | 25 | 0.5 | Q1D×1 |
| | | SRN | | | | |
| 3 | 3 | Doxorubicin- | oral | 50 | 1 | Q1D×1 |
| | | SRN | | | | |
| 4 | 3 | Doxorubicin- | oral | 75 | 1.5 | Q1D×1 |
| | | SRN | | | | |
| 5 | 3 | Doxorubicin- | oral | 100 | 2 | Q1D×1 |
| | | SRN | | | | |
| 6 | 3 | Blanck SRN | oral | 0 | none | Q1D×1 |
| | | (control) | | | | |
| 7 | 3 | Excipients | oral | 0 | none | Q1D×1 |
| | | control | | | | |

Toxicological Signs Monitoring

After oral administrations, toxicological signs were monitored (mortality, type of effects, length of effects) in the following hours. Then, mice behaviour and toxicological signs were monitored every day during the next 3 days, and then regularly until the end of the study (mice sacrifice).

Body Weight Monitoring

The body weight of animals were recorded just before treatment, then every day during the 3 days following oral administration, then regularly until the end of the study.

7.2. Results

Relative body weight (RBW) as a function of time indicates that Doxorubicine-SRN is well tolerated (see FIG. 5).

Moreover, it was observed that mice urine were orange colored when treated with doxorubicine transdrug. As orange is the color of doxorubicin-SRN, it indicates that doxorubicin go through the intestine barrier. As doxorubicin alone is poorly bioavailable when orally administered (about 5%), this indicates that an improved bioavailability of doxorubicin is expected when formulated as doxorubicin-SRN.

### Example 8

### Paclitaxel-SRN Administered by Oral Route

Paclitaxel-SRN is a new formulation of paclitaxel intended for oral administration.

8.1. Materials and Methods

For groups of rat were treated with radiolabeled paclitaxel as follow:

| | | | Paclitaxel Dose |
|---|---|---|---|
| Group | Treatment | Route | (mg/kg) |
| 1 | Paclitaxel-SRN | oral | 5 |
| 2 | Cyclodextrin-SRN | oral | 10 |
| 3 | Solution Paclitaxel - | oral | 10 |
| | SRN. (Cremophor | | |
| | EL/Ethanol) | | |

Blood samples of animals were removed 10 min, 30 min 1 H, 2 H, 3 H, 4 H, 6 H and 8 H after oral administration. Radiolabeled paclitaxel was followed by liquid scintigraphy.

8.2. Results

Major results from these studies were that Paclitaxel-SRN at a 5 mg/kg dose of paclitaxel present the same AUC values than Taxol-like solution (i.e. Solution Paclitaxel-SRN. (Cremophor EL/Ethanol)) at a two times higher dose. Interest was that this new formulation of paclitaxel was quite twice more efficient than Taxol for oral purpose, and did not need any anti-Pgp agent such as cyclosporin A for example, to enhance bioavailability.

### Example 9

### Docetaxel Solubilization

9.1. Increase of Apparent Solubility of Docetaxel with the use of Cyclodextrins

While the use of the cyclodextrins is currently one of most widely employed approaches for increasing the aqueous solubility of poorly water-soluble active substances, the data on docetaxel solubility when using different cyclodextrins described in the literature are often incomplete. Thus, inventors studied diagrams of docetaxel solubility when using different cyclodextrins such as: HP-β-CD, Me-β-CD, SBE-β-CD, α-CD and γ-CD.

9.2. Mixtures of Cyclodextrins

As shown by molecular modelling studies, it seems possible that a molecule of docetaxel might interact simultaneously with at least two cyclodextrins. Moreover, the cavities of β-cyclodextrin or γ-cyclodextrin could prove to be better adapted to one or another of the groups likely to interact. Thus, the association of different cyclodextrins constitutes an interesting strategy. Nevertheless, no work in this area has been reported in the state of the art.

Based on inventor's hypotheses, different proportions of each cyclodextrin were chosen on the one hand to obtain a total cyclodextrin concentration of 40% m/m, and on the other hand depending on their maximum solubility. In fact, Me-β-CD shows optimal solubility at 10.2% w/w and γ-CD at between 10.2% and 16% w/w.

The results displayed in Table below show the efficacy of these mixtures in terms of solubilisation. Indeed, the mixture of (HP-β-CD/Me-β-CD) at proportions of (30.6/10.2)% w/w increases docetaxel solubility 722 times in relation to docetaxel solubility in the phosphate buffer. The mixture of (HP-β-CD/γ-CD) at (30.6/10.2)% w/w allows docetaxel solubility to increase from (1.9±0.1) μg/mL to (1425±47) μg/mL. Similarly, the mixture of (Me-β-CD/γ-CD) at (10.2/10.2)% w/w increases the solubility of the active substance from (1.9±0.1) μg/mL to (1388±149) μg/mL. Regarding mixtures of three cyclodextrins, solubility is significantly improved when compared to docetaxel alone, but lower when compared to mixtures of two cyclodextrins.

TABLE
Effect of cyclodextrin mixtures on the solubility of docetaxel

| | Solubility of docetaxel | Solubility of | |
|---|---|---|---|
| | at the CD percentages | docetaxel in | |
| Formulations | used (μg/mL) | mixture (μg/mL) | F |
| (HP-β-CD/Me- | 30.6% HP-β-CD: 1048 ± 119 | 1372 ± 204 | 722 |
| β-CD) | 10.2% Me-β-CD: 1064 ± 236 | | |
| (30.6/10.2)% | | | |
| (HP-β-CD/ | 30.6% HP-β-CD: 1048 ± 119 | 1425 ± 47 | 750 |
| γ-CD) | 10.2% γ-CD: 79 ± 3 | | |
| (30.6/10.2)% | | | |
| (Me-β-CD/ | 10.2% Me-β-CD: 1064 ± 236 | 1388 ± 149 | 730 |
| γ-CD) | 10.2% γ-CD: 79 ± 3 | | |
| (10.1/10.2)% | | | |
| (HP-β-CD/ | 20.4% HP-β-CD: 972 ± 228 | 1212 ± 59 | 637 |
| Me-β- | 10.2% Me-β-CD: 1064 ± 236 | | |
| CD/γ-CD) | 10.2% γ-CD: 79 ± 3 | | |
| (20.4/10.2/ | | | |
| 10.2)% | | | |
| Control^{a} | | 1.9 ± 0.1 | 0 |

F: factor of docetaxel solubility increase compared to control

These cyclodextrins used alone improve the apparent solubility of docetaxel. When mixed, they improve it through a synergistic phenomenon, as the simultaneous action of the cyclodextrins produces an effect that is considerably greater than the sum of the isolated effects of each of the two cyclodextrins.

9.3. CONCLUSION

One of the barriers to oral absorption of docetaxel lies in the poor solubility of this substance in digestive fluids. In this work, the inventors have applied a cyclodextrines-based strategies aimed at improving docetaxel solubility.

The original approach of associating cyclodextrins of different types, particularly gamma and beta-cyclodextrins allowed this solubility to increase by a factor of up to 750.

On the practical level, work accomplished has made it possible to considerably improve the apparent solubility of docetaxel, which probably represents one of the keys to its effective oral administration.

1. Nanoparticles comprising at least one chemotherapeutic agent as an active ingredient, at least one polymer and at least one cyclic oligosaccharide capable of complexing said chemotherapeutic agent, said nanoparticles being for therapeutic oral administration.

2. A method for the treatment and/or the prevention of cancer comprising administering a nanoparticle according to claim 1.

3. Nanoparticles according to claim 1, wherein said polymer is chosen from the poly(alkylcyanoacrylate) group in which the alkyl group, linear or branched, comprises 1 to 12 twelve carbon atoms.

4. Nanoparticles according to claim 1, wherein said polymer is a poly(isohexylcyanoacrylate).

5. Nanoparticles according to claim 1, wherein said cyclic oligosaccharide capable of complexing the active ingredient is a cyclodextrin.

6. Nanoparticles according to claim 1, wherein said cyclodextrin is hydroxypropyl-β-cyclodextrin and/or Randomly Methylated-β-cyclodextrin and/or Methylated-β-cyclodextrin, and/or γ-cyclodextrin.

7. Nanoparticles according to claim 1, wherein they further comprise at least one pharmaceutically acceptable stabilizing agent, chosen from tensioactive or surfactive agent.

8. Nanoparticles according to claim 7, wherein said tensioactive or surfactive agent is a poloxamer.

9. Nanoparticles according to claim 1, wherein said chemotherapeutic agent is a topoisomerase inhibitor, an anthracycline, a spindle poison plant alkaloid, an alkylating agent, an anti-metabolite or other chemotherapeutic agent, or their pharmaceutically acceptable salts.

10. Nanoparticles according to claim 9, wherein said topoisomerase inhibitor is camptothecin derivative and preferentially irinotecan, SN-38 or topotecan, or their pharmaceutically acceptable salts.

11. Nanoparticles according to claim 9, wherein said anthracycline is doxorubicine or their pharmaceutically acceptable salts.

12. Nanoparticles according to claim 9, wherein said spindle poison plant alkaloid is paclitaxel, docetaxel or their pharmaceutically acceptable salts.

13. Nanoparticles according to claim 1 comprising:
at least one chemotherapeutic agent chosen from irinotecan, doxorubicine, paclitaxel or docetaxel or their pharmaceutically acceptable salts,
poly(isohexylcyanoacrylate);
Poloxamer 188; and
hydroxypropyl-β-cyclodextrin and/or rameb methylated-β-cyclodextrin and/or methylated-β-cyclodextrin and/or 65 -cyclodextrin.

14. Nanoparticles according to claim 1, wherein said treatment includes the administration of one or more further anticancer agent.

15. Medicament comprising at least one nanoparticle according to claim 1 in a pharmaceutically acceptable vehicle, said medicament being for oral administration.

16. Medicament comprising at least one nanoparticle according to claim 1 in a pharmaceutically acceptable vehicle for the treatment and/or the prevention of cancer, said treatment and/or prevention comprising the oral administration of said medicament.

17. A formulation of the nanoparticles comprising at least one chemotherapeutic agent according to claim 1, said formulation comprising:
said nanoparticles in solution or suspension in water, in a concentration of 0.5 to 10 mg/ml equivalent of said chemotherapeutic agent,
0.5 to 5% of a cryoprotector agent.

18. The formulation according to claim 17, comprising:
IRN-SRN in water, in a concentration of 1 to 1.5 mg/ml equivalent of IRN;
1% glucose.

19. Lyophilized nanoparticles comprising said chemotherapeutic agent according to claim 1.

20. Lyophilized nanoparticles according to claim 19 for oral administration, for the treatment and/or prevention of cancer.

21. A process of lyophilizing the nanoparticles according to claim 1, said process comprising the following steps:
Step 1: freezing the formulation comprising said nanoparticles in solution or suspension in water, in a concentration of 0.5 to 10 mg/ml equivalent of said chemotherapeutic agent, and 0.5 to 5% of a cryoprotector agent;
Step 2: primary drying said freezed formulation;
Step 3: secondary drying said primary dried freezed formulation.

22. The method according to claim 2, wherein said polymer is chosen from the poly(alkylcyanoacrylate) group in which the alkyl group, linear or branched, comprises 1 to 12 twelve carbon atoms.

23. The method according to claim 2, wherein said polymer is a poly(isohexylcyanoacrylate).

24. The method according to claim 2, wherein said cyclic oligosaccharide capable of complexing the active ingredient is a cyclodextrin.

25. The method according to claim 2, wherein said cyclodextrin is hydroxypropyl-β-cyclodextrin and/or Randomly Methylated-β-cyclodextrin and/or Methylated-β-cyclodextrin, and/or γ-cyclodextrin.

26. The method according to claim 2, wherein they further comprise at least one pharmaceutically acceptable stabilizing agent, chosen from tensioactive or surfactive agent.

27. The method according to claim 2, wherein said tensioactive or surfactive agent is a poloxamer.

28. The method according to claim 2, wherein said chemotherapeutic agent is a topoisomerase inhibitor, an anthracycline, a spindle poison plant alkaloid, an alkylating agent, an anti-metabolite or other chemotherapeutic agent, or their pharmaceutically acceptable salts.

29. The method according to claim 2, wherein said topoisomerase inhibitor is camptothecin derivative and preferentially irinotecan, SN-38 or topotecan, or their pharmaceutically acceptable salts.

30. The method according to claim 2, wherein said anthracycline is doxorubicine or their pharmaceutically acceptable salts.

31. The method according to claim 2, wherein said spindle poison plant alkaloid is paclitaxel, docetaxel or their pharmaceutically acceptable salts.

32. The method according to claim 2 comprising:
at least one chemotherapeutic agent chosen from irinotecan, doxorubicine, paclitaxel or docetaxel or their pharmaceutically acceptable salts,
poly(isohexylcyanoacrylate);
Poloxamer 188; and
hydroxypropyl-β-cyclodextrin and/or rameb methylated-β-cyclodextrin and/or methylated-β-cyclodextrin and/or γ-cyclodextrin.

33. The method according to claim 2, wherein said treatment includes the administration of one or more further anticancer agent.
